(19) United States
(12) Patent Application Publication
(10) Pub. No.: US 2025/0308680 A1
(43) Pub. Date: Oct. 2, 2025

(54) MEDICAL PROFILE EMERGENCY INFORMATION SHARING

(71) Applicant: Alarm.com Incorporated, Tysons, VA (US)

(72) Inventors: Adam Brandfass, Pittsburgh, PA (US); Mike Roth, Webster Groves, MO (US)

(21) Appl. No.: 19/084,269

(22) Filed: Mar. 19, 2025

Related U.S. Application Data
(60) Provisional application No. 63/573,197, filed on Apr. 2, 2024.

(51) Int. Cl. G16H 40/20; G16H 40/67

(52) U.S. Cl. CPC G16H 40/20; G16H 40/67

(57) ABSTRACT

Methods, systems, and apparatus, including computer programs encoded on computer storage media, for transmitting, during a medical emergency of a person, medical data of the person to a first responder or another responding individual. One of the methods includes detecting, by a first device of a person in a medical emergency, a second device that is within a threshold distance of the first device; and in response to detecting the second device and based on the medical emergency, transmitting, to the second device, medical data for the person.

### CROSS-REFERENCE TO RELATED APPLICATION

This application claims the benefit of U.S. Provisional Application No. 63/573,197, filed Apr. 2, 2024, the contents of which are incorporated by reference herein.

### BACKGROUND

Some people may experience an emergency, e.g., a medical emergency, when they are out and about. Other people who may help with the emergency can include first responders or good Samaritans who happen to be nearby.

Alarm systems can be used to detect emergencies, such as emergencies related to objects (e.g., detected by fire and carbon monoxide alarms, home intruder alarms, or car alarms) and emergencies related to humans (e.g., detected by medical equipment or medical implants). The alarm systems can sound an alarm, for example, when a sensor detects a change in an environment. The change in environment can be related to the detection of foreign matter (e.g., smoke, heat, water, or carbon monoxide), a vibration (e.g., detected by a car alarm), or a vital sign in a patient (e.g., heart rate, breathing rate, or temperature). Changes can be triggered, for example, when a detected or measured value exceeds (or falls below) a predetermined threshold. Some emergencies can cause the alarm systems to automatically contact someone who can respond to the emergency.

### SUMMARY

In general, one aspect of the subject matter described in this specification can be embodied in methods that include the actions of detecting, by a first device of a person in a medical emergency, a second device that is within a threshold distance of the first device; and in response to detecting the second device and based on the medical emergency, transmitting, to the second device, medical data for the person.

In general, some aspects of the subject matter described in this specification can be embodied in methods that include the actions of in response to a first device detecting that a second device is within a threshold distance of the first device, receiving, by the second device and from the first device of a person in a medical emergency, medical data for the person. In response to receiving the medical data, presentation can be caused of at least some of the medical data on a display of the second device.

Other implementations of this aspect include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations, the method can include determining, in response to detecting the second device, whether the second device is authenticated to receive the medical data. Transmitting the medical data for the person can be responsive to determining that the second device is authenticated to receive the medical data.

In some implementations, determining whether the second device is authenticated to receive the medical data can include: transmitting, using a first instance of an application executing on the first device, an authentication request message to the second device; in response to transmitting the authentication request message, receiving, from a second instance of the application executing on the second device, an authentication response message; and determining whether the authentication response message is valid and the second device is authenticated to receive the medical data.

In some implementations, determining whether the second device is authenticated to receive the medical data can include determining whether an identifier for the second device is included on a list of approved recipient devices. In some implementations, transmitting the medical data for the person can be responsive to determining that the identifier for the second device is included on a list of approved recipient devices.

In some implementations, transmitting the medical data can include broadcasting the medical data to a plurality of devices including the second device.

In some implementations, detecting the second device can include: receiving application programming interface (API) information that includes emergency vehicle location data; and predicting, using the emergency vehicle location data, that the second device is for a first responder.

In some implementations, the method can include providing, in response to predicting that the second device is for a first responder, an audible notification that help is on the way.

In some implementations, the method can include: in response to the first device detecting that the second device is within the threshold distance of the first device, receiving, from the first device, a request for presentation of a notification by the second device that indicates that medical data is available for presentation; presenting, by the second device, the notification that includes a user interface element that triggers a request for the medical data; receiving input that indicates selection of the user interface element and triggers a request for the medical data; and requesting, from the first device, the medical data. In some implementations, receiving, from the first device, the medical data can be responsive to requesting the medical data.

In some implementations, requesting the medical data can include: authenticating the second device for access to the medical data; and receiving the medical data is responsive to successfully completing the authentication of the second device for access to the medical data.

In some implementations, the method can include in response to the first device detecting that the second device is within the threshold distance of the first device, transmitting, to a first instance of an application executing on the first device and by a second instance of the application executing on the second device, authentication information to enable the first instance of the application to determine whether the second instance of the application is authenticated. Receiving the medical data can be responsive to the first instance of the application determining that the second instance of the application is authenticated.

In some implementations, the method can include transmitting, by the second device and to the first device, an identifier for the second device. Receiving the medical data can be responsive to transmitting the identifier for the second device to the first device.

In some implementations, the method can include receiving, at the second device and prior to receiving the request for presentation, a push notification.

In some implementations, the medical data can identify the person, the geographic location of the person, and information about the medical emergency.

This specification uses the term “configured to” in connection with systems, apparatus, and computer program components. That a system of one or more computers is configured to perform particular operations or actions means that the system has installed on it software, firmware, hardware, or a combination of them that in operation cause the system to perform those operations or actions. That one or more computer programs is configured to perform particular operations or actions means that the one or more programs include instructions that, when executed by data processing apparatus, cause the apparatus to perform those operations or actions. That special-purpose logic circuitry is configured to perform particular operations or actions means that the circuitry has electronic logic that performs those operations or actions.

The subject matter described in this specification can be implemented in various implementations and may result in one or more of the following advantages.

The techniques of the present disclosure can provide the technical advantage of making vital health information more quickly, e.g., immediately, available to others during a person's medical emergency. For example, the vital health information about the person can be provided automatically to a device of a first responder who is responding to the medical emergency, or to a device of a good Samaritan who happens to be nearby and is capable of providing at least a limited response until first responders arrive. The vital health information can include the name of the person, the person's medical information (e.g., blood type, current vital statistics, medical history, or other on-file medical information), and contact information (e.g., physicians, family, and insurance). Having the vital health information available more quickly and automatically can eliminate the need (e.g., by the first responder) to rely on asking other people in the vicinity (who may not know) specific details of past and present medical information for the person, dig through a longer file for the person to determine the medical information, or both. Providing this information can be useful in case the person is incapacitated because of the medical emergency.

In some examples, the systems and methods described in this specification can reduce or eliminate the need of the first responder to contact other people or agencies for medical information, which can cause a delay in learning vital information associated with the person and performing an emergency response. In the case of a medical emergency related to allergies, for example, medical information associated with the person can be used to initiate instructions to the first responder (or good Samaritan) to locate (and potentially administer) a nearby EPIPEN or allergy-related medications, determine which medications are not appropriate for the person, e.g., when the person is allergic to the medication, or both. Some situations that may benefit from the actions of a good Samaritan can involve diabetes-related emergencies and epilepsy-related emergencies (e.g., seizures). For example, a message can be displayed on the good Samaritan's phone stating that it is believed that the person is having an epileptic seizure, followed by a description of actions to take in response to the epileptic seizure before first responders arrive.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts an example of an environment with a medical data sharing system.

FIG. 2A is a flow diagram of a process for transmitting medical data for a person having a medical emergency from the person's device to a first responder's device.

FIG. 2B is a flow diagram of a process for receiving medical data at a first responder's device from a device of a person having an emergency.

FIG. 3 is a block diagram of a computing system that can be used in connection with computer-implemented methods described in this specification.

Like reference numbers and designations in the various drawings indicate like elements.

### DETAILED DESCRIPTION

An emergency response system can integrate, and provide communications between, applications (“apps”) installed on mobile devices of first responders (e.g., EMT personnel), mobile devices of good Samaritans, and mobile devices of people who may experience a medical emergency. The apps can be configured to make available, during a person's medical emergency, information about the person including the name of the person, the person's medical information (e.g., blood type, current vital statistics, on-file medical information), and contact information (e.g., physicians, family, and insurance). In some examples, information can be provided to the person during the medical emergency, including displayed and/or audible messages indicating that help is on the way, and an estimated arrival time of one or more first responders who have been notified of the medical emergency and are on the way.

During an emergency situation, a responder device of a person responding to the emergency might not have relevant information about the people involved in the emergency, e.g., a person having a heart attack or another medical condition, might not be able to quickly surface the relevant information, or a combination of both. This may cause the person responding to the emergency to perform non-optimal care that might otherwise be performed if the relevant information were available, spend too much time searching for the relevant information, or a combination of both.

An emergency response system can integrate, and provide communications between, applications (or “apps”) installed on mobile devices of first responders (e.g., EMT personnel), mobile devices of other people, e.g., good Samaritans, and mobile devices of people who may experience a medical emergency. The apps can be configured to make available, during a person's medical emergency, information about the person including the name of the person, the person's medical information (e.g., blood type, current vital statistics, medical history, or other on-file medical information), and contact information (e.g., physicians, family, and insurance).

During an emergency, whether medical or otherwise, the app on a mobile device can produce a display on the device, such as by triggering a push notification to automatically display health information for a person when it is determined that the person is having the medical emergency. This can occur in response to the mobile device receiving data for the health information from the person's mobile device. The health information can include information from a health card stored on the person's mobile device, information provided during the medical emergency by monitoring agency (e.g., configured to communicate with the apps on the mobile devices of the person, first responders, and good Samaritans), or both. In some examples, the push notifications can be generated (e.g., by a monitoring agency or the person's mobile device) and provided to a first responder's device or a device of another person when such a device is detected to be in the vicinity of the person, e.g., within a threshold distance of the person's device.

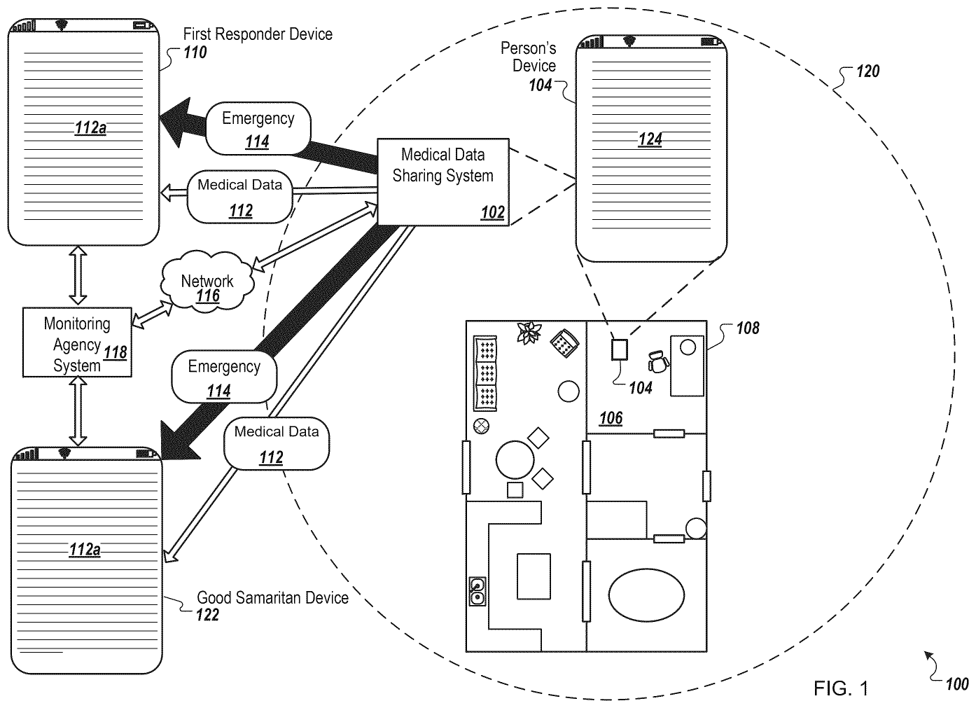

FIG. 1 depicts an example of an environment 100 with a medical data sharing system 102. The medical data sharing system 102 can be implemented, for example, as an application (or “app”) installed on a first device. In some examples, the medical data sharing system 102 can be implemented on multiple devices, e.g., as part of an overall alarm system or that includes other appropriate devices such as cloud computing or server-side devices.

For example, the first device can be a person's device 104 belonging to a person experiencing, or who might experience, a medical emergency. The emergency may be occurring, e.g., in an office 106 at the person's house 108, on the street or at another location.

The first device can detect that a second device (e.g., a first responder device 110) is within a threshold distance of the first device. The threshold distance can be any appropriate distance. In some implementations, the threshold distance can be defined by a wireless communication protocol (e.g., cellular or BLUETOOTH) available for communication between the two devices, such as to identify potential first responder devices within a five-mile radius (e.g., for cellular) or a fifty-meter radius (e.g., BLUETOOTH). In response to detecting the second device and based on the medical emergency, medical data 112 for the person can be transmitted by the first device to the second device (e.g., first responder device 110). In general, information about an emergency 114 is broadcast to the second device.

The medical data sharing system 102 can include (or be integrated with) a person monitoring system that includes a capability to monitor a person. For example, the person monitoring system can detect when changes occur in vital signs of the person and determine that the person is experiencing a medical emergency. As a result, the person monitoring system can detect when a medical emergency is likely occurring, e.g., when the person is potentially having a medical emergency, and provide a message to the medical data sharing system 102 about the potential medical emergency.

In some examples, although the person may be having a medical emergency, there may not be one or more first responders close enough to respond to the medical emergency within a reasonable time, even if the first responders have been dispatched. In such situations, the medical data sharing system 102 can communicate with the device 122 of a good Samaritan, e.g., living in the same building or nearby building, or otherwise in the general vicinity of the person having the medical emergency. Examples of good Samaritans include neighbors, building superintendents, and staff working in a building (e.g., an assisted living facility, hotel, or shopping mall) but who are not first responders, e.g., who are not currently working or not first responders in general.

When a medical emergency occurs, the medical data 112 is transmitted to one or more of first responder device(s) 110 and good Samaritan device(s) 122. Some or all of the transmitted medical data 112 can be displayed on the devices 110 and 122, as displayed medical data 112a. Timing of the transmission can have minimal delay, e.g., less than a threshold amount such that the transmission can be immediate, such as for good Samaritans already in the area of the medical emergency, or delayed, e.g., more than the threshold amount, such as for first responders responding to the medical emergency who were further away from a location of the medical emergency when the medical emergency started and have arrived within a threshold range of the person having the medical emergency.

The medical data 112 can be any appropriate type of data. In some implementations, the data can include information that is not specifically medical information, but may be useful when responding to an emergency. For example, the data can include non-medical information such as emergency contact information, a list of primary care physicians and insurance providers, timeline events, or a combination of these. A timeline of events can identify where the person was going, had recently been, or both; recent events experienced by the person; information about the person's situation and location; or a combination of these. The timeline information can include specific dates and times associated with each event.

In some implementations, the medical data sharing system 102 can broadcast a signal to all devices within a threshold distance 120 of the person's device 104. In this way, information about the emergency 114 can be broadcast more widely so as to increase a likelihood that a response to the emergency 114 can occur. The broadcast signal that is sent to the devices can result in a notification that is presented by the devices. In these implementations, the medical data sharing system 102 can require authentication by a device before the device is provided access to the medical data 112. Instructions for completing the authentication can be included in the notification.

The medical data sharing system 102 (or system 102) is an example of a system implemented as computer programs on one or more computers in one or more locations, in which the systems, components, and techniques described in this specification are implemented. The devices 104, 110, and 122 can include personal computers, mobile communication devices, and other devices that can send and receive data over a network 116. The network 116, such as a cellular (phone) network, a local area network (“LAN”), wide area network (“WAN”), the Internet, or a combination thereof, connects the devices 104, 110, and 122, the monitoring agency system 118, and the system 102. In some examples, the network 116 might connect only two devices, e.g., using a short-range wireless technology such as BLUETOOTH. The system 102 can use a single computer or multiple computers operating in conjunction with one another, including, for example, a set of remote computers deployed as a cloud computing service.

In some implementations, a monitoring agency system 118 can facilitate registration by people who wish to use the medical data sharing system 102. People who wish to be monitored, or provide assistance, e.g., a good Samaritan, during medical emergencies can register with the monitoring agency system 118. As a result of the registration, the monitoring agency system 118 can cause download and installation of the medical data sharing system 102 application on corresponding devices for the people. Part of the registration process for the people can include identifying lists of approved recipient devices that the person approves for transferring medical data during a medical emergency. The lists of approved recipient devices can also include device identifiers of the person's family, friends, neighbors, or other people. First responders can register with the monitoring agency system 118, e.g., the have the medical data sharing system 102 application downloaded and installed on their devices, to register identifiers of their devices and to designate a geographic area to which they would respond to medical emergencies. In some implementations, the monitoring agency system 118 can provide customized registration options, e.g., that allow full medical data to be provided to first responders while good Samaritans are limited to less information.

The system 102 can include several different functional components, including a system for monitoring the person (e.g., the person's vital signs), the medical data sharing application, or a combination of both. The different functional components can include one or more data processing apparatuses, can be implemented in code, or a combination of both. For instance, each of the different functional components can include one or more data processors and instructions that cause the one or more data processors to perform the operations discussed herein.

The various functional components of the system 102 can be installed on one or more computers as separate functional components or as different modules of a same functional component. For example, the different functional components of the system 102 can be implemented as computer programs installed on one or more computers in one or more locations that are coupled to each through a network (e.g., network 116). In cloud-based systems for example, these components can be implemented by individual computing nodes of a distributed computing system.

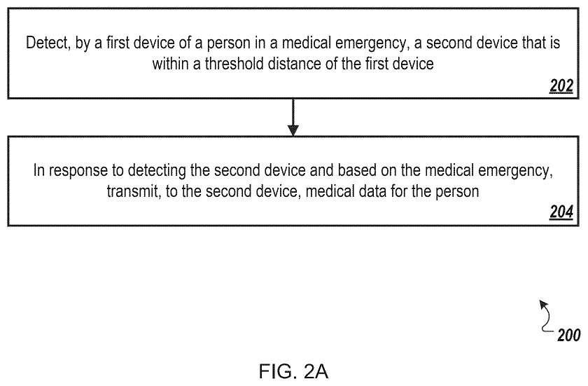

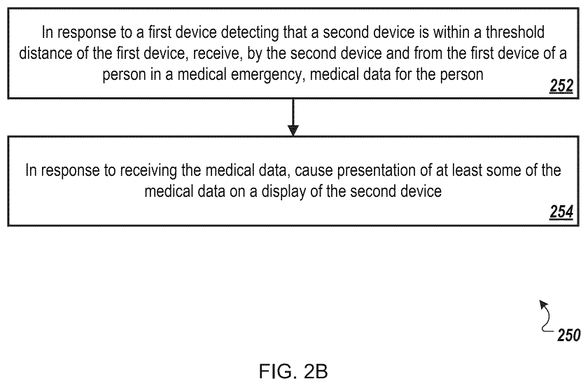

FIG. 2A is a flow diagram of a process 200 for transmitting medical data for a person having a medical emergency from the person's device to a first responder's device. For example, the process 200 can be used by the medical data sharing system 102 from the environment 100.

A second device is detected that is within a threshold distance of a first device of a person in a medical emergency (202). The second device can be, for example, the first responder device 110 that is within a threshold distance 120 (e.g., a radius of five miles or fifty-meters) of the person's device 104. The threshold distance can be defined by whatever wireless communication protocol is used between the devices 104, 110, and 122 and can vary based on the types of devices for the person and the first responder.

In some implementations, detecting the second device can include detecting that the second device is a device that belongs to a first responder. For example, the first device (e.g., the person's device 104) can receive application programming interface (API) information that includes emergency vehicle location data, such as geographic location coordinates. The geographic location information can include, over a short time sequence, more than one set of specific geographic location coordinates so that a direction-of-travel (e.g., toward a geographic location of the person) is indicated. Based on this information, including using the using the emergency vehicle location data, the medical data sharing system 102 can predict that the second device is for a responder.

In some implementations, the person can receive a notification that help is on the way. For example, in response to predicting that the second device is for a first responder, the medical data sharing system 102 can provide an audible notification (e.g., a voice message) that help is on the way. The notification can include, for example, identification of the type(s) of first responder(s) en route to the person's emergency, an estimated time of arrival, or both. The notification can include a statement that the first responders have been briefed on the person's emergency and the person's medical history. In some examples, a user interface 124 on the person's device can display a message indicating that help is on the way.

Medical data for the person is transmitted to the second device in response to detecting the second device and based on the medical emergency (204). For example, the medical data sharing system 102 can send, to the first responder device 110, vital sign information for the patient, including heart rate, breathing rate, temperature, blood glucose level, any other medical data that the medical data sharing system 102 determines to be applicable to the medical emergency, or a combination of these. The medical data can include information about the medical history of the person, e.g., that the person is a known diabetic, has a history of heart issues, or is known to be a person with epilepsy. Information sent with the medical data can include a location of the person, e.g., in the living room of the first floor or a bedroom of the second floor, instructions for entering the person's building (e.g., to use a specific door to enter), or a combination of both.

In some implementations, an authentication process can be performed to authenticate the second device before allowing the medical data to be sent to the second device. For example, in response to detecting the second device (e.g., within a threshold distance 120 of five miles or 50 meters), the medical data sharing system 102 can determine whether the second device (e.g., the first responder device 110) is authenticated to receive the medical data. In response to determining that authentication is successful, the medical data sharing system 102 can transmit the medical data 112 for the person to the second device.

The authentication process can be any appropriate type of authentication process. In some implementations, the authentication process can include sending and receiving authentication request messages. For example, determining whether the second device (e.g., the first responder device 110) is authenticated to receive the medical data can include transmitting, using a first instance of an application executing on the first device (e.g., the person's device 104), an authentication request message to the second device. In response to transmitting the authentication request message, the medical data sharing system 102 can receive, from a second instance of the application executing on the second device, an authentication response message. The medical data sharing system 102 can determine whether the authentication response message is valid (e.g., matches a code or a security question) and the second device is authenticated to receive the medical data.

In some implementations, determining whether the second device is authenticated to receive the medical data can include determining whether an identifier for the second device is included on a list of approved recipient devices. The medical data sharing system 102 can transmit the medical data for the person in response to determining that the identifier for the second device is included on a list of approved recipient devices (e.g., identified by the person while registering for the medical data sharing system 102 service). The identifier for the second device can be, for example, a media access control (MAC) address (e.g., 00-B0-xx-xx-xx-01) or some other hardware address, physical address, or identifier (ID) that is used to identify electronic devices on a network.

The application used in the authentication process can be a security application, with first and second instances of the security application residing on the devices of persons who have registered with the medical data sharing system 102 app and service, and for first responders who have registered to respond to emergencies of such persons.

In some implementations, medical data that is transmitted can be transmitted to and received by multiple devices. In an example, transmitting the medical data can include broadcasting the medical data 112 to a several devices, e.g., including the second device, resulting in the medical data 102 being received by multiple first responder devices 110. The multiple-device transmittal can occur, for example, if two or more of a fire truck, an ambulance, and a police car respond to a dispatched call for an emergency.

The order of operations in the process 200 described above is illustrative only. Some of operations 202-204 can include sub-operations that are performed, for example, in different orders or by components of the monitoring agency system 118. For example, if some of the operations performed by the first device 104 are not performable during an emergency because of a lost connection between the person's device and the first responder's device, then the monitoring agency system 118 can perform the operations or different operations as needed to communicate with, and provide emergency information, e.g., medical data, to, the first responder's device in order to complete the emergency response.

In some implementations, the process 200 can include additional operations, fewer operations, or some of the operations can be divided into multiple operations. For example, additional operations can be added for people to register with the monitoring agency system 118, such as to subscribe to the service and to identify lists of list of approved recipient devices, or to set parameters defining when their device is to trigger an emergency response (e.g., heart rates above N beats per minute).

FIG. 2B is a flow diagram of a process 250 for receiving medical data at a first responder's device from a device of a person having an emergency. For example, the process 250 can be used by the medical data sharing system 102 within the environment 100. The process 250 is described from the perspective of a first responder device, as compared to the process 200 which is described from the perspective of a device of the person having the emergency. At least some of the medical data can be presented in a user interface on the first responder's device, e.g., limited to pertinent information that is associated with the type of medical emergency that is occurring when the medical emergency type is available.

Medical data for a person in a medical emergency is received by a second device from a first device of a person (252). For example, medical data 112 is received in response to the first device (e.g., the person's device 104) detecting that the second device (e.g., the first responder device 110) is within a threshold distance of the first device, such as while the first responder is responding to the emergency and has arrived or almost arrived at the location of the medical emergency. The medical data identifies the person having the medical emergency. In some examples, the medical data can identify the geographic location of the person, information about the medical emergency, or both. The geographic location can be shared earlier with the monitoring agency system, or already available to the first responder in some way, e.g., when the monitoring agency system has a profile for the person.

In some implementations, process 250 can include a series of inputs involving a user interface at the second device. For example, in response to the first device (e.g., the person's device 104) detecting that the second device (e.g., the first responder device 110) is within the threshold distance of the first device, a request for presentation of a notification can be received by the second device from the first device that indicates that medical data is available for presentation. The notification, including a user interface element that triggers a request for the medical data, is presented by the second device (e.g., to the first responder). Input can be received that indicates selection of the user interface element and trigger a request for the medical data. The medical data is requested from the first device, from memory, e.g., when the medical data was included in the request for presentation of the notification, or a combination of both. As such, the medical data that is received from the first device is received in response to requesting the medical data.

In some implementations, a push notification can be received at the second device (e.g., the first responder device 110) prior to receiving the request for presentation. The push notification, for example, can alert the application on the first responder's device that data for an emergency situation is about to be received, is available for access, or a combination of both. The application, if unopen at the time, can then open and be ready for receiving data, presenting data, or both.

In some implementations, the process 250 includes authentication. For example, requesting the medical data can include authenticating the second device for access to the medical data. In response to successfully completing the authentication of the second device (e.g., the first responder device 110) for access to the medical data, the medical data can be received by the second device. For example, when the medical data 112 is received on the first responder device 110, the information can be displayed as displayed medical data 112a. Examples of authentication processes are described in more detail above.

In some implementations, the process 250 includes a distance threshold test. For example, in response to the first device (e.g., the person's device 104) detecting that the second device (e.g., the first responder device 110) is within the threshold distance of the first device, authentication information is transmitted. The authentication information can be transmitted by a second instance of the application executing on the second device to a first instance of an application executing on the first device. The authentication information enables the first instance of the application to determine whether the second instance of the application is authenticated. Receiving the medical data at the second device is then responsive to the first instance of the application determining that the second instance of the application is authenticated.

In some implementations, an identifier for the second device is transmitted, by the second device to the first device (e.g., the first responder device 110). Receiving the medical data is then responsive to transmitting the identifier for the second device to the first device. The transmission of the identifier is part of an authentication process to ensure that the second device is associated with an entry on the approved list of responder devices process. The approved listed can be for the first device, the monitoring agency system, another appropriate system, or a combination of two or more of these.

At least some of the medical data is caused to be presented on a display of the second device in response to receiving the medical data (404). The medical data that can be presented identifies the person (e.g., including the person's first name so that the first responder can use their name in conversation when talking to the person) and medical information for the person, e.g., medical history information. The medical data can identify the geographic location of the person (e.g., map coordinates to a location of the person, and a specific room or other location), and information about the medical emergency (e.g., specific details of a suspected heart attack other medical condition).

The order of operations in the process 250 described above is illustrative only. Some of operations 252-254 can include sub-operations that are performed, for example, in different orders or by components of the monitoring agency system 118. For example, if some of the actions expected to be completed by the first device 104 (e.g., the first responder device 110) are not performable during an emergency because of a lost connection between various devices, then the monitoring agency system 118 can perform operations needed to communicate with, and provide emergency information to, the first responder device in order to complete the emergency response.

In some implementations, the process 250 can include additional operations, fewer operations, or some of the operations can be divided into multiple operations. For example, additional operations can be added for first responder devices (or groups of first responders devices in an agency, such as an ambulance service, police precinct, or a fire district) to register with the monitoring agency system 118, such as to provide a list identifiers for devices under their control (to be used by people to create lists of approved recipient devices).

For situations in which the systems discussed here collect personal information about users, or may make use of personal information (e.g., by transmitting medical data 112 to the first responder device 110), the users (e.g., persons potentially having a medical emergency) may be provided (e.g., through preferences selected when registering with the monitoring agency) with an opportunity to control which personal and medical information is transmitted and to whom and in what situations. In addition, certain data may be anonymized in one or more ways before it is stored or used, so that personally identifiable information (PII) is removed. For example, a user's identity may be anonymized so that no PII can be determined for the user, or a user's geographic location may be generalized where location information is obtained (such as to a city, ZIP code, or state level), so that a particular location of a user cannot be determined. Thus, the user may have control over how information is collected about him or her and used. Exceptions to generalizing and sharing location information and PII of the person may exist when warranted in response to a medical emergency.

In this specification, the term “database” is used broadly to refer to any collection of data: the data does not need to be structured in any particular way, or structured at all, and it can be stored on storage devices in one or more locations. A database can be implemented on any appropriate type of memory.

In this specification the term “engine” is used broadly to refer to a software-based system, subsystem, or process that is programmed to perform one or more specific functions. Generally, an engine will be implemented as one or more software modules or components, installed on one or more computers in one or more locations. In some instances, one or more computers will be dedicated to a particular engine. In some instances, multiple engines can be installed and running on the same computer or computers.

A number of implementations have been described. Nevertheless, it will be understood that various modifications can be made without departing from the spirit and scope of the disclosure. For example, various forms of the flows shown above can be used, with operations re-ordered, added, or removed.

Implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible non-transitory program carrier for execution by, or to control the operation of, a data processing apparatus. Alternatively, or in addition, the program instructions can be encoded on an artificially-generated propagated signal, e.g., a machine-generated electrical, optical, or electromagnetic signal, that is generated to encode information for transmission to a suitable receiver apparatus for execution by a data processing apparatus. One or more computer storage media can include a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can be or include special purpose logic circuitry, e.g., a field programmable gate array (“FPGA”) or an application-specific integrated circuit (“ASIC”). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can be performed by, and apparatus can also be implemented as, special purpose logic circuitry, e.g., a field programmable gate array (“FPGA”) or an application-specific integrated circuit (“ASIC”).

Computers suitable for the execution of a computer program include, by way of example, general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. A computer can be embedded in another device, e.g., a mobile telephone, a smart phone, a headset, a personal digital assistant (“PDA”), a mobile audio or video player, a game console, a Global Positioning System (“GPS”) receiver, or a portable storage device, e.g., a universal serial bus (“USB”) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a liquid crystal display (“LCD”), an organic light emitting diode (“OLED”) or other monitor, for displaying information to the user and a keyboard and a pointing device, e.g., a mouse or a trackball or a touchscreen, by which the user can provide input to the computer. Other kinds of devices can be used to provide for interaction with a user as well. For example, feedback provided to the user can be any form of sensory feedback, e.g., visual feedback, auditory feedback, or tactile feedback; and input from the user can be received in any form, including acoustic, speech, or tactile input. In some examples, a computer can interact with a user by sending documents to and receiving documents from a device that is used by the user; for example, by sending web pages to a web browser on a user's device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., a Hypertext Markup Language (“HTML”) page, to a user device, e.g., for purposes of displaying data to and receiving user input from a user device, which acts as a client. Data generated at the user device, e.g., a result of user interaction with the user device, can be received from the user device at the server.

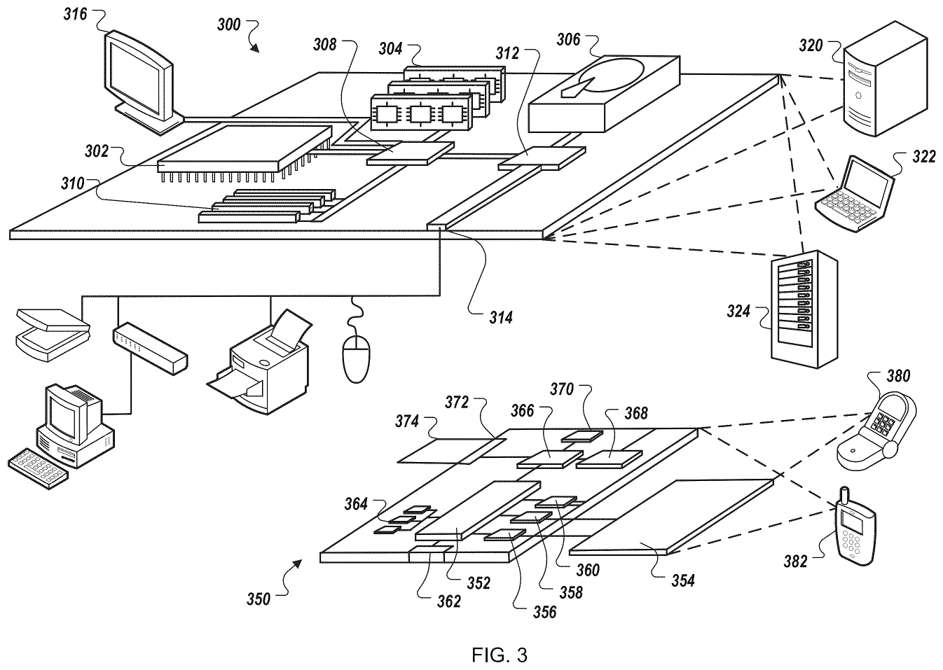

FIG. 3 is a block diagram of computing devices 300, 350 that may be used to implement the systems and methods described in this specification, as either a client or as a server or plurality of servers. Computing device 300 is intended to represent various forms of digital computers, such as laptops, desktops, workstations, personal digital assistants, servers, blade servers, mainframes, and other appropriate computers. Computing device 350 is intended to represent various forms of mobile devices, such as personal digital assistants, cellular telephones, smartphones, smartwatches, head-worn devices, and other similar computing devices. The components shown here, their connections and relationships, and their functions, are meant to be exemplary only, and are not meant to limit implementations described and/or claimed in this specification.

Computing device 300 includes a processor 302, memory 304, a storage device 306, a high-speed interface 308 connecting to memory 304 and high-speed expansion ports 310, and a low speed interface 312 connecting to low speed bus 314 and storage device 306. Each of the components 302, 304, 306, 308, 310, and 312, are interconnected using various busses, and may be mounted on a common motherboard or in other manners as appropriate. The processor 302 can process instructions for execution within the computing device 300, including instructions stored in the memory 304 or on the storage device 306 to display graphical information for a GUI on an external input/output device, such as display 316 coupled to high speed interface 308. In other implementations, multiple processors and/or multiple buses may be used, as appropriate, along with multiple memories and types of memory. Also, multiple computing devices 300 may be connected, with each device providing portions of the necessary operations (e.g., as a server bank, a group of blade servers, or a multi-processor system).

The memory 304 stores information within the computing device 300. In one implementation, the memory 304 is a computer-readable medium. In one implementation, the memory 304 is a volatile memory unit or units. In another implementation, the memory 304 is a non-volatile memory unit or units.

The storage device 306 is capable of providing mass storage for the computing device 300. In one implementation, the storage device 306 is a computer-readable medium. In various different implementations, the storage device 306 may be a floppy disk device, a hard disk device, an optical disk device, or a tape device, a flash memory or other similar solid state memory device, or an array of devices, including devices in a storage area network or other configurations. In one implementation, a computer program product is tangibly embodied in an information carrier. The computer program product contains instructions that, when executed, perform one or more methods, such as those described above. The information carrier is a computer-or machine-readable medium, such as the memory 304, the storage device 306, or memory on processor 302.

The high speed controller 308 manages bandwidth-intensive operations for the computing device 300, while the low speed controller 312 manages lower bandwidth-intensive operations. Such allocation of duties is exemplary only. In one implementation, the high-speed controller 308 is coupled to memory 304, display 316 (e.g., through a graphics processor or accelerator), and to high-speed expansion ports 310, which may accept various expansion cards (not shown). In the implementation, low-speed controller 312 is coupled to storage device 306 and low-speed expansion port 314. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 300 may be implemented in a number of different forms, as shown in the figure. For example, it may be implemented as a standard server 320, or multiple times in a group of such servers. It may also be implemented as part of a rack server system 324. In addition, it may be implemented in a personal computer such as a laptop computer 322. Alternatively, components from computing device 300 may be combined with other components in a mobile device (not shown), such as device 350. Each of such devices may contain one or more of computing device 300, 350, and an entire system may be made up of multiple computing devices 300, 350 communicating with each other.

Computing device 350 includes a processor 352, memory 364, an input/output device such as a display 354, a communication interface 366, and a transceiver 368, among other components. The device 350 may also be provided with a storage device, such as a Microdrive or other device, to provide additional storage. Each of the components 350, 352, 364, 354, 366, and 368, are interconnected using various buses, and several of the components may be mounted on a common motherboard or in other manners as appropriate.

The processor 352 can process instructions for execution within the computing device 350, including instructions stored in the memory 364. The processor may also include separate analog and digital processors. The processor may provide, for example, for coordination of the other components of the device 350, such as control of user interfaces, applications run by device 350, and wireless communication by device 350.

Processor 352 may communicate with a user through control interface 358 and display interface 356 coupled to a display 354. The display 354 may be, for example, a TFT LCD display or an OLED display, or other appropriate display technology. The display interface 356 may comprise appropriate circuitry for driving the display 354 to present graphical and other information to a user. The control interface 358 may receive commands from a user and convert them for submission to the processor 352. In addition, an external interface 362 may be provided in communication with processor 352, so as to enable near area communication of device 350 with other devices. External interface 362 may provide, for example, for wired communication (e.g., via a docking procedure) or for wireless communication (e.g., via Bluetooth or other such technologies).

The memory 364 stores information within the computing device 350. In one implementation, the memory 364 is a computer-readable medium. In one implementation, the memory 364 is a volatile memory unit or units. In another implementation, the memory 364 is a non-volatile memory unit or units. Expansion memory 374 may also be provided and connected to device 350 through expansion interface 372, which may include, for example, a SIMM card interface. Such expansion memory 374 may provide extra storage space for device 350, or may also store applications or other information for device 350. Specifically, expansion memory 374 may include instructions to carry out or supplement the processes described above, and may include secure information also. Thus, for example, expansion memory 374 may be provided as a security module for device 350, and may be programmed with instructions that permit secure use of device 350. In addition, secure applications may be provided via the SIMM cards, along with additional information, such as placing identifying information on the SIMM card in a non-hackable manner.

The memory may include for example, flash memory and/or MRAM memory, as discussed below. In one implementation, a computer program product is tangibly embodied in an information carrier. The computer program product contains instructions that, when executed, perform one or more methods, such as those described above. The information carrier is a computer-or machine-readable medium, such as the memory 364, expansion memory 374, or memory on processor 352.

Device 350 may communicate wirelessly through communication interface 366, which may include digital signal processing circuitry where necessary. Communication interface 366 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 368. In addition, short-range communication may occur, such as using a Bluetooth, WiFi, or other such transceiver (not shown). In addition, GPS receiver module 370 may provide additional wireless data to device 350, which may be used as appropriate by applications running on device 350.

Device 350 may also communicate audibly using audio codec 360, which may receive spoken information from a user and convert it to usable digital information. Audio codec 360 may likewise generate audible sound for a user, such as through a speaker, e.g., in a handset of device 350. Such sound may include sound from voice telephone calls, may include recorded sound (e.g., voice messages, music files, etc.) and may also include sound generated by applications operating on device 350.

The computing device 350 may be implemented in a number of different forms, as shown in the figure. For example, it may be implemented as a cellular telephone 380. It may also be implemented as part of a smartphone 382, personal digital assistant, or another similar mobile device.

Various implementations of the systems and techniques described here can be realized in digital electronic circuitry, integrated circuitry, specially designed ASICS (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some instances be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures, such as spreadsheets, relational databases, or structured files, may be used.

Particular implementations of the invention have been described. Other implementations are within the scope of the following claims. For example, the operations recited in the claims, described in the specification, or depicted in the figures can be performed in a different order and still achieve desirable results. In some implementations, multitasking and parallel processing may be advantageous.

1. A method comprising:
detecting, by a first device of a person in a medical emergency, a second device that is within a threshold distance of the first device; and
in response to detecting the second device and based on the medical emergency, transmitting, to the second device, medical data for the person.

2. The method of claim 1, comprising:
in response to detecting the second device, determining whether the second device is authenticated to receive the medical data,
wherein transmitting the medical data for the person is responsive to determining that the second device is authenticated to receive the medical data.

3. The method of claim 2, wherein determining whether the second device is authenticated to receive the medical data comprises:
transmitting, using a first instance of an application executing on the first device, an authentication request message to the second device;
in response to transmitting the authentication request message, receiving, from a second instance of the application executing on the second device, an authentication response message; and
determining whether the authentication response message is valid and the second device is authenticated to receive the medical data.

4. The method of claim 2, wherein:
determining whether the second device is authenticated to receive the medical data comprises determining whether an identifier for the second device is included on a list of approved recipient devices; and
transmitting the medical data for the person is responsive to determining that the identifier for the second device is included on a list of approved recipient devices.

5. The method of claim 1, wherein transmitting the medical data comprises broadcasting the medical data to a plurality of devices including the second device.

6. The method of claim 1, wherein detecting the second device comprises:
receiving application programming interface information that includes emergency vehicle location data; and
predicting, using the emergency vehicle location data, that the second device is for a first responder.

7. The method of claim 6, comprising:
providing, in response to predicting that the second device is for a first responder, an audible notification that help is on the way.

8. One or more non-transitory computer storage media encoded with instructions that, when executed by one or more computers, cause the one or more computers to perform operations comprising:
in response to a first device detecting that a second device is within a threshold distance of the first device, receiving, by the second device and from the first device of a person in a medical emergency, medical data for the person; and
in response to receiving the medical data, causing presentation of at least some of the medical data on a display of the second device.

9. The media of claim 8, the operations comprising:
in response to the first device detecting that the second device is within the threshold distance of the first device, receiving, from the first device, a request for presentation of a notification by the second device that indicates that medical data is available for presentation;
presenting, by the second device, the notification that includes a user interface element that triggers a request for the medical data;
receiving input that indicates selection of the user interface element and triggers a request for the medical data; and
requesting, from the first device, the medical data,
wherein receiving, from the first device, the medical data in responsive to requesting the medical data.

10. The media of claim 9, wherein:
requesting the medical data comprises authenticating the second device for access to the medical data; and
receiving the medical data is responsive to successfully completing the authentication of the second device for access to the medical data.

11. The media of claim 9, the operations comprising:
receiving, at the second device and prior to receiving the request for presentation, a push notification.

12. The media of claim 8, the operations comprising:
in response to the first device detecting that the second device is within the threshold distance of the first device, transmitting, to a first instance of an application executing on the first device and by a second instance of the application executing on the second device, authentication information to enable the first instance of the application to determine whether the second instance of the application is authenticated,
wherein receiving the medical data is responsive to the first instance of the application determining that the second instance of the application is authenticated.

13. The media of claim 8, the operations comprising:
transmitting, by the second device and to the first device, an identifier for the second device,
wherein receiving the medical data is responsive to transmitting the identifier for the second device to the first device.

14. The media of claim 8, wherein the medical data identifies the person, a geographic location of the person, and information about the medical emergency.

15. A system comprising one or more computers and one or more storage devices on which are stored instructions that are operable, when executed by the one or more computers, to cause the one or more computers to perform operations comprising:
detecting, by a first device of a person in a medical emergency, a second device that is within a threshold distance of the first device; and
in response to detecting the second device and based on the medical emergency, transmitting, to the second device, medical data for the person.

16. The system of claim 15, the operations comprising:
in response to detecting the second device, determining whether the second device is authenticated to receive the medical data,
wherein transmitting the medical data for the person is responsive to determining that the second device is authenticated to receive the medical data.

17. The system of claim 16, wherein determining whether the second device is authenticated to receive the medical data comprises:
transmitting, using a first instance of an application executing on the first device, an authentication request message to the second device;
in response to transmitting the authentication request message, receiving, from a second instance of the application executing on the second device, an authentication response message; and
determining whether the authentication response message is valid and the second device is authenticated to receive the medical data.

18. The system of claim 16, wherein:
determining whether the second device is authenticated to receive the medical data comprises determining whether an identifier for the second device is included on a list of approved recipient devices; and
transmitting the medical data for the person is responsive to determining that the identifier for the second device is included on a list of approved recipient devices.

19. The system of claim 15, wherein transmitting the medical data comprises broadcasting the medical data to a plurality of devices including the second device.

20. The system of claim 15, wherein detecting the second device comprises:
receiving application programming interface information that includes emergency vehicle location data; and
predicting, using the emergency vehicle location data, that the second device is for a first responder.
